(19) United States
(12) Patent Application Publication
(10) Pub. No.: US 2025/0195340 A1
(43) Pub. Date: Jun. 19, 2025

(54) CLEANSING WIPE COMPRISING HYDROPHOBIC POLYMER

(71) Applicant: L'OREAL, Paris (FR)

(72) Inventors: Ritesh Kumar SINHA, Linden, NJ (US); Hy Si BUI, Piscataway, NJ (US); Xuefei LIU, Edison, NJ (US)

(73) Assignee: L'OREAL, Paris (FR)

(21) Appl. No.: 18/544,195

(22) Filed: Dec. 18, 2023

(51) Int. Cl. A61K 8/02; A61K 8/06; A61K 8/31; A61K 8/37; A61K 8/46; A61K 8/60; A61K 8/81; A61K 8/92; A61Q 1/14

(52) U.S. Cl. CPC A61K 8/0208; A61K 8/062; A61K 8/31; A61K 8/37; A61K 8/466; A61K 8/608; A61K 8/8152; A61K 8/922; A61Q 1/14

(57) ABSTRACT

The instant disclosure is drawn to a cleansing wipe comprising: (I) an insoluble flexible substrate impregnated with a cleansing composition; and (ii) the cleansing composition. The cleansing composition includes: (a) a hydrophobic polymer formed as a reaction product of a natural or food-derived oil and a methacrylate or acrylate polymer; (b) one or more solvents capable solubilizing the hydrophobic polymer; (c) one or more surfactants; and (d) water, wherein the cleansing composition is an oil-in-water emulsion or dispersion. Methods of cleansing a surface and methods for removing makeup from skin are also described.

### FIELD OF THE DISCLOSURE

The instant case is drawn to a cleansing wipe comprising a hydrophobic polymer and methods for cleansing a substrate using the cleansing wipe.

### BACKGROUND

A wide variety of pre-moistened wipe products are available for cleansing surfaces, for example, for home, automotive, medical, industrial, and personal use. While the intended use may vary, they share several similar characteristics. For example, most cleansing wipes have a rectangular or square form, and are usually slightly larger in size than an average human hand. They are formed from a soft, thin material that may be either single-ply or multi-ply and are pre-moistened with a cleansing composition. In some cases, in addition to cleansing, they soften or soothe the surface being wiped (e.g., personal hygiene wipes for human use). In other cases, they clean and condition the surface being wiped (e.g., furniture or automotive wipes).

Cleansing wipes often are sold in plastic or other packaging such as tubes, bags, or other containers, and users extract the wipe by pulling it through a hole or slot in the packaging. Preferably, the hole or slot can be covered by a lid or other mechanism to prevent the cleansing wipes from drying. Commonly, the container or packaging is pre-loaded with a supply of wipes. Some cleansing wipes are formed from a continuous run of material, having perforations to assist in separating one wipe from another, which is conformally folded or rolled for dispensing. Other conventional wipes are individually formed and separated from one another, and usually conformally folded for dispensing. Regardless, most wipes are packaged together for dispensing in some type of sealable container, to preserve the pre-moistening and keep them from drying out.

A wide variety of cleansing wipes are commercially available to meet consumers' various needs. For example, some consumers desire scented wipes, while other consumers demand wipes that are free of all dyes, scents, or perfumes. Still other consumers desire wipes having lotions such as aloe vera, lanolin, or other materials, often with or without the addition of alcohol. Still other consumers want gentle wipes, or wipes free of any lotion, alcohol, or other additives. Regardless of the differing consumer demands, a critical element common to all cleansing wipes is the requirement that they effective cleanse. When a cleansing wipe has insufficient cleansing properties, multiple cleansing wipes are used causing unnecessary waste and time. Oils, dirt, and contaminates can be smeared around when the cleansing wipe does not efficiently and adequately lift and absorb them. Therefore, cleansing wipes that have superior cleansing properties are desirable.

### SUMMARY OF THE DISCLOSURE

The cleansing wipes of the instant disclosure are surprisingly effective for dislodging, absorbing, and remove contamination, especially oils, makeup, and greasy contaminants. The effectiveness is due, at least in part, to a unique hydrophobic polymer that improves the dispersion of the cleansing composition carried by the cleansing wipe. In addition, the hydrophobic polymer has a binding affinity for contaminates, thereby enhancing the cleansing wipes' ability to dislodge and absorb the contaminants quickly. Furthermore, the hydrophobic polymer reduces surface tension in the cleansing composition, which allows for smaller oil droplets to be dispersed throughout the composition providing robust and stable emulsions that successfully carry and deliver cleansing actives and other desirable active ingredients, for example, skin active ingredients in situations where the cleansing wipes are for cleansing or treating the skin. In view of these multiple advantages, the cleansing wipes are particularly well suited for lifting and removing makeup from the skin. The cleansing wipes typically include:
(i) an insoluble flexible substrate, which may be a woven or non-woven substrate, wherein the substrate is impregnated with a cleansing composition; and
(ii) the cleansing composition, wherein the cleansing composition comprises:
   (a) a hydrophobic polymer formed as a reaction product of a natural or food-derived oil and a methacrylate or acrylate polymer;
   (b) one or more solvents capable of solubilizing the reaction product of (a);
   (c) one or more surfactants; and
   (d) water.

The insoluble flexible substrate may be a woven or non-woven substrate and absorbs or carries the cleansing composition, i.e., the insoluble flexible substrate is impregnated with the cleansing composition. The insoluble flexible substrate may be formed from a variety of materials both natural and synthetic. Further, the insoluble flexible substrate by be a foam, a sponge, wadding, a sheet, a cloth, or a film.

The hydrophobic polymer is a reaction product of a natural or food-derived oil and an acrylate or methacrylate polymer. According to embodiments of the disclosure, however, the hydrophobic polymer is the reaction product of a natural or food-derived oil and a methacrylate polymer. The natural or food-derived oil may be a drying oil or a semi-drying oil. Nonlimiting examples include linseed oil, sunflower oil, tung oil, fish oil, cottonseed oil, soybean oil, or combinations thereof. The methacrylate polymer can be formed from methacrylate monomers, for example, monomers selected from isobutyl methacrylate, methyl methacrylate, ethyl methacrylate, n-butyl methacrylate, or combinations thereof. In a preferred embodiment, the hydrophobic polymer is formed from a natural or food-derived oil and an isobutyl methacrylate polymer and may preferably be an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly(isobutyl methacrylate) in a suitable solvent, such as, for example, 2,2,4-trimethyl-1, 3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1, 3-pentanediol monoisobutyrate.

One or more solvents capable of solubilizing the hydrophobic polymer of (a) is used to solubilize the hydrophobic polymer. A single solvent may be used or a combination of solvents, wherein the combination of solvents can solubilize the hydrophobic polymer of (a). In various embodiments, the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameter, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer of (a) include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. Polycitrnoellol acetate is particularly useful.

Surfactants include anionic surfactants, cationic surfactants, amphoteric (zwitterionic) surfactants, and nonionic surfactants. In various embodiments, the cleansing composition includes one or more anionic surfactants, and optionally, one or more nonionic surfactants. Anionic surfactants are useful for their detersive properties. They are effective cleansers and can contribute to foaming, if desired. Nonlimiting examples of anionic surfactants include glutamates, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof. In various embodiments, the cleansing composition includes one or more acyl taurate surfactants.

One or more of the surfactants may be a biosurfactant. Nonlimiting examples of biosurfactants include glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes. Preferably, at least one of the biosurfactants is a glycolipid. Nonlimiting examples of glycolipids include sophorolipids, rhamnolipids, trehalose lipids, mannosylerythritol lipids, and combinations thereof, wherein rhamnolipids are particularly preferred.

The cleansing composition includes a sizeable amount of water, which forms all or part of the aqueous phase of the cleansing composition. In addition, the aqueous phase may include one or more water soluble solvents, hydrophilic active agents, salts, etc. Similarly, the oil phase, in addition to the hydrophobic polymer, may include one or more lipophilic active agents, for example, one or more skin active agents such as ceramides, cholesterol, etc.

Also described are methods for cleansing a surface using the cleansing wipes. For example, in a preferred embodiment, the cleansing wipes are particularly useful for cleansing the skin and for removing makeup from the skin.

### DETAILED DESCRIPTION OF THE DISCLOSURE

The instant disclosure is drawn to cleansing wipes impregnated with a cleansing composition. The cleansing composition is typically an oil-in-water emulsion comprising a hydrophobic polymer. The cleansing composition provides improved cleansing properties and is surprisingly robust, versatile, and useful for carrying and delivering cosmetic ingredients, including skin active agents. The cleansing wipe typically comprises:
(i) an insoluble flexible substrate, which may be a woven or non-woven substrate, wherein the substrate is impregnated with a cleansing composition; and
(ii) the cleansing composition, wherein the cleansing composition comprises:
   (a) a hydrophobic polymer formed as a reaction product of a natural or food-derived oil and a methacrylate or acrylate polymer;
   (b) one or more solvents capable of solubilizing the hydrophobic polymer (a);
   (c) one or more surfactants; and
   (d) water.

The cleansing composition is preferably an oil-in-water emulsion. In various embodiments, the oil-in-water emulsion comprises oil droplets having an average size of from about 10 nm to about 1 μm.

The cleansing composition may optionally include a variety of additional ingredients. For example, in certain embodiments, the cleansing composition includes one or more skin active agents, vitamins, thickening agents, water soluble solvents, fatty acids, fatty alcohols, or combinations thereof. Nonlimiting examples of skin active agents includes anti-wrinkle agents, anti-inflammatory agents, depigmenting agents, skin renewal compounds, compounds that improve skin barrier function, anti-aging compounds, and the like.

Due to the cosmetic composition's stability, versatility, and multiple phases (oil/water), the cosmetic compositions may include a variety of different skin active agents. For example, the oil phase of the cosmetic composition may include one or more lipophilic skin active agents. The aqueous phase of the cosmetic composition may include one or hydrophilic skin active agents. Nonlimiting examples of useful skin active agents include ceramides, ceramide precursors, cholesterol, cholesterol sulfate, b-glucan, carob seed extract, eperua falcata extract, amino acids, niacinamide and its derivatives, hyaluronic acid & its derivatives allantoin, omega fatty acids, vitamins, vitamin precursors, retinoids including retinol, retinoic acid, tretinoin, isotretinoin, adapalene, or combinations thereof.

In a preferred embodiment, the cosmetic composition includes one or more ceramides, for example, one or more ceramides selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, and Ceramide EOdS, protein bound ceramides, phytosphingosine, sphingosine, ceramide precursors, or combinations thereof.

The cleansing composition is useful for cleansing a surface and is particularly useful for cleansing the skin. In a preferred embodiment, the cleansing composition is useful in methods for removing makeup from the skin, for example, the skin of the face.

### (i) Insoluble Flexible Substrate

An “insoluble” substrate is a substrate that does not dissolve or readily break apart upon immersion in water and immersion in the cleansing composition that the insoluble flexible substrate carries. A wide variety of materials can be used. The following nonlimiting characteristics are desirable: (i) sufficient wet strength for use, (ii) sufficient abrasiveness, (ill) sufficient loft and porosity, (iv) sufficient thickness, and (v) appropriate size. The term “flexible” with respect to the insoluble flexible substrate means that the substrate is easily deformed and pliable. A typical cotton cleansing rag is an example of a flexible substrate. The insoluble flexible substrate is absorbent, that is, able to take liquid in through the surface and hold it.

The insoluble flexible substrate may be a woven or non-woven substrate and absorbs or carries the cleansing composition, i.e., the insoluble flexible substrate is impregnated with the cleansing composition. The insoluble flexible substrate may be formed from a variety of materials both natural and synthetic. Further, the insoluble flexible substrate by be a foam, a sponge, wadding, a sheet, a cloth, or a film.

Nonlimiting examples of suitable insoluble flexible substrates which meet the above criteria include nonwoven substrates, woven substrates, hydro-entangled substrates, air entangled substrates and the like. Preferred embodiments employ nonwoven substrates since they are economical and readily available in a variety of materials. By nonwoven is meant that the layer is comprised of fibers which are not woven into a fabric but rather are formed into a sheet, particularly a tissue. The fibers can either be random (i.e., randomly aligned) or they can be carded (i.e. combed to be oriented in primarily one direction). Furthermore, the nonwoven substrate can be composed of a combination of layers of random and carded fibers.

The insoluble substrate may comprise one or more layers and may be selected from woven materials, nonwoven materials, foams, sponges, wadding, sheets, cloth, or films. It may be a nonwoven or woven substrate based on fibers of natural origin (linen, wool, cotton, silk, bamboo fibers) or of synthetic origin (cellulose derivatives, viscose, polyvinyl derivatives, polyesters such as polyethylene terephthalate, polyolefins such as polyethylene (PET) or polypropylene, polyamides such as nylon, acrylic derivatives), and mixtures thereof such as viscose/PET, polylactic acid (PLA), viscose/polylactic acid (viscose/PLA).

Nonwoven substrates may be comprised of a variety of materials both natural and synthetic. By natural is meant that the materials are derived from plants, animals, insects, or byproducts. By synthetic is meant that the materials are obtained primarily from various man-made materials or from material that is usually a fibrous web comprising any of the common synthetic or natural textile-length fibers, or mixtures thereof.

Nonlimiting examples of useful natural materials include silk fibers, keratin fibers, and cellulosic fibers. Nonlimiting examples of keratin fibers include those selected from wool fibers, camel hair fibers, and the like. Nonlimiting examples of cellulosic fibers include those selected from wood pulp fibers, cotton fibers, hemp fibers, jute fibers, flax fibers, and mixtures thereof.

Nonlimiting examples of useful synthetic materials include those selected from acetate fibers, acrylic fibers, cellulose ester fibers, modacrylic fibers, polyamide fibers, polyester fibers, polyolefin fibers, polyvinyl alcohol fibers, rayon fibers and mixtures thereof. Examples of some of these synthetic materials include acrylics such as ACRILAN®, CRESLAN®, and the acrylonitrile-based fiber, ORLON®; cellulose ester fibers such as cellulose acetate, ARNEL®, and ACELE®; polyamides such as Nylons (e.g., Nylon 6, Nylon 66, and Nylon 610); polyesters such as FORTREL®, KODEL®, and DACRON®; polyolefins such as polypropylene, polyethylene; polyvinyl acetate fibers and mixtures thereof.

Woven substrates include knitted substrates and are often made from threads or strings of natural or synthetic material woven into a wipe. Nonlimiting examples include fabric and cloth. For example, cotton, polyester, and the like.

Nonwoven substrates made from natural materials consist of webs or sheets are commonly formed on a fine wire screen from a liquid suspension of the fibers. Substrates made from natural materials can be obtained from a wide variety of commercial sources. Nonlimiting examples of suitable commercially available paper layers useful herein include AIRTEX®, an embossed airlaid cellulosic layer having a base weight of about 71 gsy, available from James River Corporation, Green Bay, Wis.; and WALKISOFT®, an embossed airlaid cellulosic having a base weight of about 75 gsy, available from Walkisoft U.S.A., Mount Holly, N.C.

Useful nonwoven substrates made from synthetic material can also be obtained from a wide variety of commercial sources. Nonlimiting examples of suitable non-woven layer materials useful herein include HFE-40-047, an apertured hydroentangled material containing about 50% rayon and 50% polyester, and having a basis weight of about 43 grams per square yard (gsy), available from Vertec, Inc., Walpole, Mass.; HEF 140-102, an apertured hydro-entangled material containing about 50% rayon and 50% polyester, and having a basis weight of about 56 gsy, available from Veratec, Inc., Walpole, Mass.; NOVENET® 149-191, a thermo-bonded grid patterned material containing about 69% rayon, about 25% polypropylene, and about 6% cotton, and having a basis weight of about 100 gsy, available from Veratec, Inc., Walpole, Mass.; HEF NUBTEX® 149-801, a nubbed, apertured hydro-entangled material, containing about 100% polyester, and having a basis weight of about 70 gsy, available from Veratec, Inc. Walpole, Mass.; KEYBAK® 951V, a dry formed apertured material, containing about 75% rayon, about 25% acrylic fibers, and having a basis weight of about 43 gsy, available from Chicopee Corporation, New Brunswick, N.J.; KEYBAK® 1368, an apertured material, containing about 75% rayon, about 5% polyester, and having a basis weight of about 39 gsy, available from Chicopee Corporation, New Brunswick, N. J.; DURALACE® 1236, an apertured, hydro-entangled material, containing about 100% rayon, and having a basis weight from about 40 gsy to about 115 gsy, available from Chicopee Corporation, New Brunswick, N.J.; DURALACE® 5904, an apertured, hydro-entangled material, containing about 100% polyester, and having a basis weight from about 40 gsy to about 115 gsy, available from Chicopee Corporation, New Brunswick, N.J.; SONTARO® 8868, a hydro-entangled material, containing about 50% cellulose and about 50% polyester, and having a basis weight of about 60 gsy, available from Dupont Chemical Corp.

The insoluble substrates have comprise two or more layers, each having a different texture and abrasiveness. The differing textures can result from the use of different combinations of materials or from the use of a substrate having a more abrasive side for exfoliation and a softer, absorbent side for gentle cleansing. In addition, separate layers of the substrate can be manufactured to have different colors, thereby helping the user to further distinguish the surfaces.

The substrate can be of any size and shape suitable for the purpose. Moreover, it generally has an area of between 0.005 m² and 0.1 m², preferably between 0.01 m² and 0.05 m².

The impregnation rate of the composition on the substrate is generally from 100 to 1000%, preferably from 250 to 700% by weight of the substrate. Techniques for impregnating substrates with compositions are well known in the art and are all applicable to the present case. In general, the impregnating composition is optionally heated and added to the substrate by one or more techniques including dipping, coating, spraying, etc.

A number of wipes are typically stored together in a single pouch, for example, anywhere from 1 to 500, from 1 to 100, or from 5 to 50 single wipes may be stored within a dispensing pouch or container, preferably a moisture impermeable pouch or container. During storage and between dispensing, the pouch or container is preferably resealable. Single wipe containing pouches may also be employed.

The amount of impregnating composition (cleansing composition) relative to the substrate may range from about 20:1 to 1:20, preferably from 10:1 to about 1:10 and more preferably from about 2:1 to about 1:2 by weight. The cleaning wipe may be loaded with at least 1, 1.5 or 2 grams of the cleaning composition, as described herein, per gram of dry substrate, but typically not more than 5 grams per gram.

### (a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may preferably include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer has an oil component and a polymer component, typically reacted in a solvent. In a preferred embodiment, the hydrophobic polymer is a reaction product of linseed oil and poly(isobutyl methacrylate) in a solvent, such as 2, 2, 4-trimethyl-1,3-pentanediol-monoisobutyrate.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component. The oil component typically forms about 72% to 77%, or most preferably, 74.62%, of the hydrophobic polymer (e.g., linseed oil/isobutyl methacrylate).

The polymer component may be derived from α and β-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, or methyl methacrylic acid. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or combinations thereof.

Preferably the polymer is poly(isobutyl methacrylate). In particular, the polymer percentage may be from about 23% to about 28%, or about 25.28%, of the hydrophobic polyer, e.g., poly(linseed oil/isobutyl methacrylate). The hydrophobic polymer is a reaction product typically formed in a liquid solvent able to dissolve or dilute the polymer component and the reaction product, i.e., the hydrophobic polymer (poly(oil/polymer)). The solvent, or diluent should generally comprise any liquid or mixture of liquids that is able to dissolve or dilute hydrophobic polymer. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2, 2, 4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties.

The amount of hydrophobic polymer in the cleansing composition will vary but is often in an amount from about 0.1 to about 15 wt. %, based on the total weight of the cleansing composition. In further embodiments, the total amount of the hydrophobic polymer in the cleansing composition is from about 0.1 to about 12 wt. %, about 0.1 to about 10 wt. %, about 0.1 to about 8 wt. %, about 0.1 to about 5 wt. %, about 0.1 to about 3 wt. %, about 0.5 to about 15 wt. %, about 0.5 to about 12 wt. %, about 0.5 to about 10 wt. %, about 0.5 to about 8 wt. %, about 0.5 to about 5 wt. %, about 0.5 to about 3 wt. %, about 1 to about 15 wt. %, about 1 to about 12 wt. %, about 1 to about 10 wt. %, about 1 to about 8 wt. %, about 1 to about 5 wt. %, about 1 to about 3 wt. %, about 2 to about 15 wt. %, about 2 to about 12 wt. %, about 2 to about 10 wt. %, about 2 to about 8 wt. %, about 2 to about 5 wt. %, based on the total weight of the cleansing composition.

### (b) Solvent Capable of Solubilizing (a)

The oil phase of the cleansing composition includes the hydrophobic polymer of (a) dissolved in one or more solvents capable of solubilizing the hydrophobic polymer of (a). The one or more solvents may include a solvent used to in the reaction to form the hydrophobic polymer, e.g., 2, 2, 4-trimethyl-1,3-pentanediol-monoisobutyrate. The one or more solvents may be a single solvent or a plurality of solvents. For example, in various embodiments, solvents capable of solubilizing the hydrophobic polymer of (a) have a dispersion component (D), a polar component (P), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (P), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5.}

The solvent may be an oil. The term “oil” is intended to mean a non-aqueous compound, non-miscible in water, liquid at 25° C. and atmospheric pressure (760 mmHg; 1.013×10⁵ Pa). The solvent may be a non-silicone oil (e.g., an oil that does not contain silicon atoms, and in particular does not contain Si—O groups). Non-limiting examples of the one or more solvents (b) include caprylic/capric triglyceride, isopropyl myristate, and polycitronellol acetate. The solvent may include acetone. The solvent may include oleic acid. The solvent may include an oleic acid containing oil (such as a vegetable oil). Table 1, below, shows values of D, P, and H, as well as Ra values for the allowable range as well as the preferred range, for several solvents.

TABLE 1

| | | | | Ra | Ra |
|---|---|---|---|---|---|
| | | | | (Allowable | (Preferred |
| Solvent (b) | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl | 15.9 | 2.1 | 2.8 | 10.70 | 9.41 |
| Myristate | | | | | |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol | 16.4 | 3 | 4.2 | 9.02 | 7.76 |
| Acetate | | | | | |
| Acetone | 15.5 | 10.4 | 7 | 8.61 | 7.38 |
| Oleic Acid | 16 | 2.8 | 6.2 | 7.27 | 5.98 |

In some embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the solvent may include macadamia nut oil, and may be free, or substantially free, of other vegetable oils.

For purposes of the instant disclosure, the one or more solvents capable of solubilizing the hydrophobic polymer of (a) may not individually solubilize the hydrophobic polymer of (a) but when combined with other solvents, the combination solubilizes the hydrophobic polymer of (a). Thus, when referring to a total amount of one or more solvents capable of solubilizing the hydrophobic polymer of (a), the inclusion of all solvents that in combination solubilize the hydrophobic polymer of (a) is intended, even if one or more solvents in the combination do not individually solubilize the hydrophobic polymer of (a).

Nonlimiting examples of solvents for solubilizing the hydrophobic polymer of (a), individually or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer of (a) are selected from caprylic/capric triglyceride, polycitronellol acetate, isododecane, and mixtures thereof. In another preferred embodiment, at least one of the one or more solvents capable of solubilizing the hydrophobic polymer of (a) is polycitronellol acetate.

Nonlimiting solvents that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

The total amount of the one or more solvents capable of solubilizing the hydrophobic polymer of (a) in the cleansing composition will vary and can be adjusted depending on hydrophobic/lipophilic properties desired and the intended target to be cleansed. In certain embodiments, it is desirable to have a smaller oil phase and therefore a smaller amount of the one or more solvents is needed. In other embodiments, it is useful for have a larger oil phase, such that a higher amount of the one or more solvents is needed.

It can be useful to have a large aqueous phase. In this case, the total amount of the one or more solvents capable of solubilizing the hydrophobic polymer (a) may be from about 0.05 to about 10 wt. %, based on the total weight of the cleansing composition. In further embodiments, the total amount of the one or more solvents capable of solubilizing the hydrophobic polymer of (a) in the cleansing composition is from about 0.05 to about 8 wt. %, about 0.05 to about 5 wt. %, about 0.05 to about 3 wt. %, about 0.1 to about 10 wt. %, about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, or about 0.5 to about 3 wt. %, based on the total weight of the cleansing composition.

In other instances, it is useful to have a large oil phase. In this case, the total amount of the one or more solvents capable of solubilizing the hydrophobic polymer (a) maybe in an amount from about 1 to about 60 wt. %, based on the total weight of the cleansing composition. In further embodiments, the cleansing composition may include from about 1 to about 50 wt. %, about 1 to about 40 wt. %, about 1 to about 30 wt. %, about 2 to about 60 wt. %, about 2 to about 50 wt. %, about 2 to about 40 wt. %, about 2 to about 30 wt. %, about 5 to about 60 wt. %, about 5 to about 50 wt. %, about 5 to about 40 wt. %, about 5 to about 30 wt. %, about 10 to about 60 wt. %, about 10 to about 50 wt. %, about 10 to about 40 wt. %, or about 10 to about 30 wt. %, based on the total weight of the cleansing composition.

### (c) Surfactant

For purposes of the instant disclosure, the term “surfactant” includes emulsifiers and detergents. Surfactants, or surface-active agents, are compounds that lower the surface tension between two liquids or between a liquid and a solid. Surfactants are amphiphilic, meaning that they contain hydrophilic (water-loving) head groups and hydrophobic (water-hating, or oil-loving) tails. Surfactants adsorb at the interface between oil and water, thereby decreasing the surface tension.

For purposes of the instant disclosure, an “emulsifier” is a surfactant that stabilizes emulsions. Emulsifiers coat droplets within an emulsion and prevent them from coming together, or coalescing. An “emulsion” is a mixture of two or more liquids, with or without an emulsifier, that are normally immiscible. One of the liquids, the “dispersed phase,” forms droplets in the other liquid, the “continuous phase.”

A “detergent” is a surfactant that has cleaning properties in dilute solutions and is typically anionic.

The surfactants can be anionic, cationic, amphoteric (zwitterionic), or nonionic. Preferably, the emulsions of the instant case include one or more surfactants selected from anionic surfactants, amphoteric (zwitterionic) surfactants, nonionic surfactants, or mixtures thereof. In various embodiments, the emulsions are preferably free or essentially free from cationic surfactants. In other embodiments, the emulsions include one or more cationic surfactants. In preferred embodiments, the emulsions contain one or more biosurfactants, one or more anionic surfactants, optionally, one or more nonionic surfactants, or mixtures thereof.

In a preferred embodiment, the compositions of the instant disclosure include a plurality of surfactants, wherein the plurality of surfactants include one or more biosurfactants and one or more surfactants other than the one or more biosurfactants. In further embodiments, the compositions of the instant disclosure include one or more biosurfactants, one or more anionic surfactants, and optionally, one or more nonionic surfactants.

The total amount of the one or more surfactants in the cleansing composition will vary but is typically from about 0.5 to about 20 wt. %, based on the total amount of the cleansing composition. In further embodiments, the total amount of the one or more surfactants in the cleansing composition is from about 0.5 to about 15 wt. %, about 0.5 to about 12 wt. %, about 0.5 to about 8 wt. %, about 0.5 to about 6 wt. %, about 1 to about 20 wt. %, about 1 to about 15 wt. %, about 1 to about 12 wt. %, about 1 to about 10 wt. %, about 1 to about 8 wt. %, about 1 to about 6 wt. %, about 2 to about 20 wt. %, about 2 to about 15 wt. %, about 2 to about 12 wt. %, about 2 to about 10 wt. %, about 2 to about 8 wt. %, about 2 to about 6 wt. %, about 3 to about 20 wt. %, about 3 to about 15 wt. %, about 3 to about 12 wt. %, about 3 to about 10 wt. %, about 3 to about 8 wt. %, about 3 to about 6 wt. %, about 4 to about 20 wt. %, about 4 to about 15 wt. %, about 4 to about 12 wt. %, about 4 to about 10 wt. %, about 4 to about 8 wt. %, or about 4 to about 6 wt. %, based on the total weight of the cleansing composition.

### Biosurfactant

The compositions of the instant disclosure may optionally include one or more biosurfactants. Biosurfactants are amphiphilic molecules, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are environmentally friendly, biodegradable, and non-toxic and may be classified into high and low molecular weight biosurfactants. Low molecular weight biosurfactant efficiently lower surface and interfacial tension, and high molecular weight biosurfactants are more effective as emulsion-stabilizing agents. Examples of low molecular weight biosurfactants include glycolipids, such as rhamnolipids, sophorolipids, lipopeptidesm, and trehalolipids. These low molecular weight biosurfactants have hydrophilic heads comprised of sugar units linked glycosidically with hydrophobic non-polar parts. Examples of high molecular weight biosurfactants include polysaccharides, lipopolysaccharides, proteins, and lipoproteins. Polysaccharide-based biosurfactant can be classified into sorbitan esters, sucrose esters and glucose-based surfactants that include alkyl polyglycosides and fatty acid glucamides.

Nonlimiting examples of biosurfactants include liptopeptides such as surfactin; fatty acids and phospholipids, polymeric matrix biosurfactants; particulate biosurfactants; and bacterial biosurfactants composed of polysaccharides, proteins, lipopolysaccharides, lipoproteins or complex mixtures of these biopolymers.

Nonlimiting examples of commercially available biosurfactants include alkyl polyglycoside available under the trademark ECOSENSE® 3000 from Dow Chemical®; D-glucopyranose, oligomeric, decyl octyl glycosides available under the trademark GLUCOPON® 215 from BASF Corporation®; rhamnolipids available under the trademark REWOFERM® SL ONE from Evonik®; D-Glucitol, 1-deoxy-1-(methylamino)-, N-coco acyl derivatives available under the trademark GLUCOTAIN® from Clariant®; rhamnolipids from Jeneil Biotech®, and BioLoop® surfactants from Lankem® Ltd.

In one embodiment, the microbial biosurfactant is a glycolipid such as rhamnolipids (RLP), sophorolipids (SLP), trehalose lipid or mannosylerythritol lipid (MEL). The biosurfactants can be added in purified form or can be present in the microbe-based composition as a result of microbial growth. The biosurfactant may be a sophorolipid. In some embodiments, the biosurfactant can also be a lipopeptide, such as surfactin, and/or a rhamnolipid.

In some embodiments, a blend of biosurfactants is present. Preferably the blend comprises a rhamnolipid, and optionally one or both of a mannosylerythritol lipid, a surfactin or a sophorolipid. In a preferred embodiment, the microbe is a non-pathogenic strain of Pseudomonas. Preferably, the strain is a producer of rhamnolipid (RLP) biosurfactants.

Other microbial strains including, for example, other fungal strains capable of accumulating significant amounts of, for example, glycolipid-biosurfactants can be used in accordance with the subject invention. Biosurfactants useful according to the present invention include mannoprotein, beta-glucan and other metabolites that have bio-emulsifying and surface/interfacial tension-reducing properties.

In various embodiments, the one or more biosurfactants are selected from surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. In one embodiment, the biosurfactants are selected from one or more glycolipids such as, for example, rhamnolipids, rhamnose-d-phospholipids, trehalose lipids, trehalose dimycolates, trehalose monomycolates, mannosylerythritol lipids, cellobiose lipids, ustilagic acid and/or sophorolipids.

In various embodiments, the biosurfactant has an anionic character, for example, sophorolipids, trehalolipid and rhamnolipids. Preferable are the mono-rhamnolipids and di-rhamnolipids. The preferred alkyl chain length is from C₈ to C₁₂. The alkyl chain may be saturated or unsaturated.

The term “rhamnolipids” includes compounds of the general formula (II) and salts thereof,

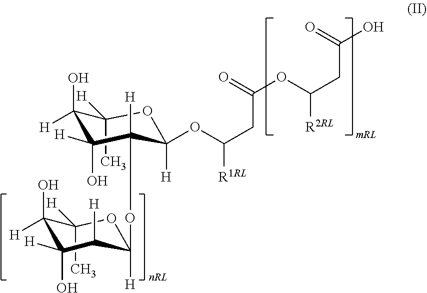

wherein,

mRL=2, 1 or 0,

5 nRL=1 or 0,

R^{1RL} and R^{2RL}=are independently organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CHs where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. I-3-{I-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where nRL=1.

The term “mono-rhamnolipid” in the context of the present invention is understood to mean compounds of the general formula (II) or salts thereof, where nRL=0.

Distinct rhamnolipids are abbreviated according to the following nomenclature: “diRL-CXCY” are understood to mean di-rhamnolipids of the general formula (II), in which one of the residues R^{1RL} and R^{2RL}═(CH2)ₒ—CH₃ where o=X-4 and the remaining residue R¹ or R²—(CH₂)ₒ—CH₃ where o=Y-4.

“monoRL-CXCY” are understood to mean mono-rhamnolipids of the general formula (II), in which one of the residues R^{1RL} and R^{2RL}═(CH.sub.2).sub.ₒ-CH.sub.₃ where o=X-4 and the remaining residue R^{1RL} or R^{2RL}═(CH₂)ₒ—CH₃ where o=Y-4. The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “: Z”, this signifies that the respective residue R^{1RL} and/or R^{2RL} is equal to an unbranched, unsubstituted hydrocarbon residue having X-3 or Y-3 carbon atoms having Z double bonds.

Methods for preparing the relevant rhamnolipids are disclosed, for example, in EP2786743 and EP2787065, which are incorporated herein by reference in their entirety. Rhamolipids can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been Improved for higher rhamnolipid titres by genetical modification can also be used in the context of the instant invention; such cells have for example been disclosed by Lei et al. in BIOTECHNOL LETT. 2020 June; 42 (6): 997-1002, which is incorporated herein by reference in its entirety. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789, which are incorporated herein by reference in their entirety.

Rhamnolipids produced by Pseudomonas aeruginosa are commercially available from Jeneil Biotech Inc., e.g. under the tradename ZONIX®, from Logos Technologies (technology acquired by Stepan), e.g. under the tradename NATSURFACT®, from Biotensidon GmbH, e.g. under the tradename RHAPYNAL®, from AGAER technologies, e.g. under the name R90, R95, R95Md, R95Dd, from Locus Bio-Energy Solutions and from Shanghai Yusheng Industry Co. Ltd., e.g. under the tradename BIO-201 GLYCOLIPIDS®.

The total amount of the one or more biosurfactants in the cleansing composition, if present, will vary but is typically from about 0.1 to about 20 wt. %, based on the total weight of the cleansing composition. In further embodiments, the total amount of the one or more biosurfactants in the cleansing composition is from about 0.1 to about 15 wt. %, about 0.1 to about 10 wt. %, or about 0.1 to about 5 wt. %, based on the total weight of the cleansing composition. In a further embodiment, the total amount of the one or more biosurfactants in the cleansing composition is from about 0.5 to about 20 wt. %, about 0.5 to about 15 wt. %, about 0.5 to about 10 wt. %, or about 0.5 to about 5 wt. %, based on the total weight of the cleansing composition. In yet a further embodiment, the total amount of the one or more biosurfactants in the cleansing composition is from about 1 to about 20 wt. %, about 1 to about 15 wt. %, about 1 to about 10 wt. %, or about 1 to about 5 wt. %, based on the total weight of the cleansing compos. In a preferred embodiment, the total amount of the one or more biosurfactants in the cleansing compos is from about 2 to about 20 wt. %, about 2 to about 15 wt. %, about 2 to about 10 wt. %, about 2 to about 5 wt. %, about 3 to about 20 wt. %, about 3 to about 15 wt. %, about 3 to about 10 wt. %, about 3 to about 5 wt. %, about 2 to about 8 wt. %, about 2 to about 6 wt. %, about 3 to about 8 wt. %, or about 3 to about 6 wt. %, based on the total weight of the cleansing composition.

### Anionic Surfactants

In various embodiment, the compositions of the instant disclosure include one or more anionic surfactants. Common popular anionic surfactants include sodium lauryl sulfate and sodium laureth ether sulfate, which may be used. The one or more anionic surfactants, if present, may also be non-sulfate anionic surfactants. Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. In some cases, however, acyl taurates are preferred and therefore the one or more non-sulfate anionic surfactants include at least one acyl taurate. In other cases, acyl isethionates are preferred and therefore the one or more non-sulfate anionic surfactants include at least one acyl isethionate.

In yet other cases, a combination of acyl taurates and acyl isethionates may be used. Thus, the cleansing compositions may include two or more non-sulfate anionic surfactants comprising anionic surfactants selected from acyl taurates, acyl isethionates, or combinations thereof.

The total amount of the one or more anionic surfactants in the cleansing composition, if present, will vary, but is typically in an amount from about 0.01 to about 10 wt. %, based on the total weight of the cleansing composition. In further embodiments, the cleansing composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more anionic surfactants, based on the total weight of the cleansing composition.

Non-limiting examples of non-sulfate anionic surfactants are provided below.

### (a) Acyl Isethionates

Non-limiting examples of useful acyl isethionates include those of formula (III) and (IV):

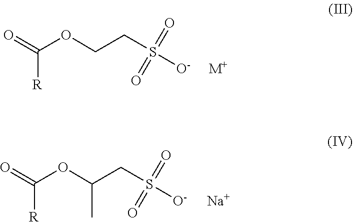

wherein R, R¹, R² and R³ are each independently selected from H or an alkyl chain having 1-24 carbon atoms, said chain being saturated or unsaturated, linear or branched, and X is COO⁻ or SO₃⁻. Although sodium is shown as the cation in formulae (III) and (IV), the cation for both formula (III) and formula (IV) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate. In some embodiments, a combination of sodium isethionate and sodium cocoyl isethionate are preferable.

### (b) Alkyl Sulfonates

Examples of alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful.

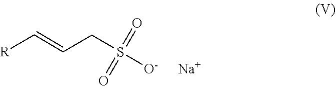

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (V) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates may be particularly preferred. A non-limiting example of a C₁₀-C₂₄ olefin sulfonate that can be used in the instant compositions is sodium C₁₄-C₁₆ olefin sulfonate.

### (c) Alkyl Sulfosuccinates

Non-limiting examples of useful sulfosuccinates include those of formula (VI):

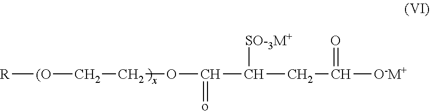

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

### (d) Alkyl Sulfoacetates

Non-limiting examples of alkyl sulfacetates includes, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### (e) Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)
wherein:
R is a hydrocarbon radical containing from about 6 to about 40 carbon atoms;
u, v and w, independently of one another, represent numbers of from 0 to 60;
x, y and z, independently of one another, represent numbers of from 0 to 13;
R′ represents hydrogen, alkyl, and
the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C₆-C₄₀ alkyl or alkenyl group or a C₁-C₄₀ alkyl phenyl group, more typically a C₈-C₂₂ alkyl or alkenyl group or a C₄-C₁₈ alkyl phenyl group, and even more typically a C₁₂-C₁₈ alkyl group or alkenyl group or a C₆-C₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉-C₁₁ Pareth-6 Carboxylic Acid, C₁₁-C₁₅ Pareth-7 Carboxylic Acid, C₁₂-C₁₃ Pareth-5 Carboxylic Acid, C₁₂-C₁₃ Pareth-8 Carboxylic Acid, C₁₂-C₁₃ Pareth-12 Carboxylic Acid, C₁₂-C₁₅ Pareth-7 Carboxylic Acid, C₁₂-C₁₅ Pareth-8 Carboxylic Acid, C₁₄-C₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

### (f) Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of acyl amino acids include those of formula (VIII):

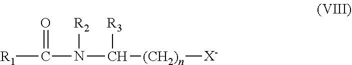

wherein R, R¹, R² and R³ are each independently selected from H or an alkyl chain having 1-24 carbon atoms, said chain being saturated or unsaturated, linear or branched, and X is COO⁻ or SO₃⁻.

### (g) Acyl Taurates

Non-limiting examples of acyl taurates include those of formula (IX):

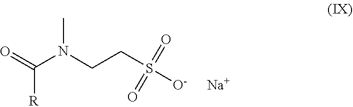

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, sodium lauroyl taurate, and sodium methyl lauroyl taurate.

### (h) Acyl Glycinates

Non-limiting examples of acyl glycinates include those of formula (X):

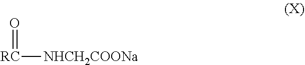

wherein R is an alkyl chain of 8 to 16 carbon atoms. Although sodium is shown as the cation in the above formula (X), the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### (i) Acyl Glutamates

Non-limiting examples of acyl glutamates include those of formula (XI):

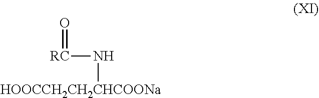

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

### (j) Acyl Sarcosinates:

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Amphoteric Surfactants

The compositions of the instant disclosure may optionally include one or more amphoteric surfactants. Nonlimiting examples of amphoteric surfactants include betaines, alkyl amphoacetates and alkyl amphodiacetates, alkyl sulltaines, alkyl amphopropionates, and combinations thereof.

The total amount of the one or more amphoteric surfactants added to the cleansing composition may be from about 0.01 to about 10 wt. %, based on the total weight of the cleansing composition. In further embodiments, the cleansing composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more amphoteric surfactants, based on the total weight of the cleansing composition.

### (a) Betaines

The one or more betaine surfactants may be in the form of a salt in the cleansing composition or before addition to the cleansing composition. The betaine surfactants may be derived from a variety of natural oils or fatty acids.

In some embodiments, exemplary useful betaines include, but are not limited to, those of the following formulae (Ia-Id):

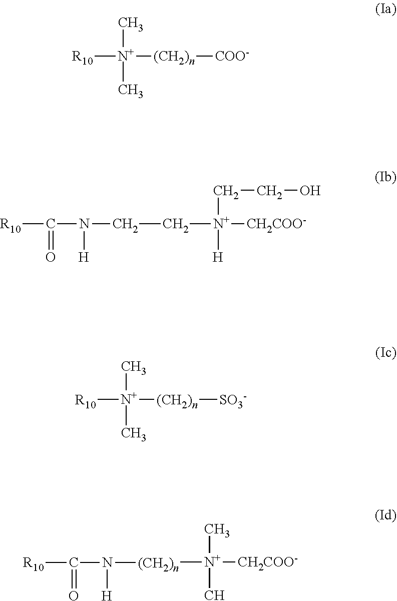

wherein:

R₁₀ is an alkyl group having from 8-18 carbon atoms; and

n is an integer from 1 to 3.

Particularly useful betaines include, for example, coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof. Typically, at least one betaine compound is selected from coco betaine, cocamidopropyl betaine, behenyl betaine, capryl/capramidopropyl betaine, and lauryl betaine, and mixtures thereof. In one embodiment, preferred betaines include coco-betaine and cocamidopropyl betaine.

### (b) Alkyl Amphoacetates and Alkyl Amphodiacetates

By way of example only, useful alkyl amphoacetates and alkyl amphodiacetates include those of Formula (Iia) or (Iib):

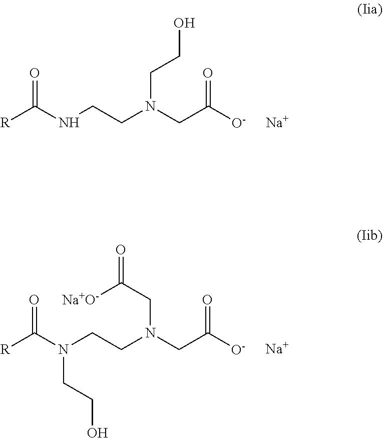

wherein R is an alkyl group having 8-18 carbon atoms.

Although sodium is shown as the cation in the above formulae, the cation may be any alkali metal ion, such as sodium or potassium, an ammonium ion, or an alkanolammonium ion such as monoethanolammonium or triethanolammonium ions. A non-limiting example is sodium lauroamphoacetate.

Additional non-limiting examples of alkyl amphoacetates and alkyl amphodiacetates include those of formula (Iic):

Ra′-CON(Z)CH2-(CH2)m′-N(B)(B′) (Iic)

wherein:
B represents —CH2CH2OX′, with X′ representing —CH2-COOH, CH2-COOZ′, —CH2CH2-COOH, —CH2CH2-COOZ′, or a hydrogen atom;
B′ represents —CH2)z-Y′, with z=1 or 2, and Y′ representing —COOH, —COOZ′, —CH2-CHOH—SO3H or —CH2-CHOH—SO3Z′;
m′ is equal to 0, 1 or 2;
Z represents a hydrogen atom or a hydroxyethyl or carboxymethyl group;
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanol-amine, diisopropanolamine or triisopropanolamine, 2-amino-2-methyl-1-propanol, 2-amino-2-methyl-1,3-propanediol and tris(hydroxy-methyl)aminomethane; and
Ra′ represents a (C10-C30)alkyl or alkenyl group of an acid Ra′COOH preferably present in hydrolyzed linseed oil or coconut oil, an alkyl group, in particular a C17 alkyl group, and its iso form, or an unsaturated C17 group.

Exemplary compounds of formula (Ic) include (C8-C20)alkylamphoacetates and (C8-C20)alkylamphodiacetates, such as disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylomphodipropionate, lauroamphodipropionic acid, or cocoamphodipropionic acid. For example, disodium cocoamphodiacetate supplied by Rhodia under the name MIRANOLI C₂M can be used.

### (c) Alkyl Sulltaines

Non-limiting examples of alkyl sultaines include hydroxyl sultaines of the following formula (Iid)

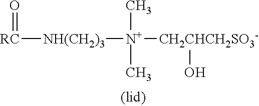

wherein R is an alkyl group having 8-18 carbon atoms. More specific examples include, but are not limited to cocamidopropyl hydroxysultaine, lauryl hydroxysultaine, and a mixture thereof.

### (d) Alkyl Amphopropionates

Non-limiting examples of alkyl amphopropionates include cocoamphopropionate, cornamphopropionatecaprylamphopropionate, cornamphopropionate, caproamphopropionate, oleoamphopropionate, isostearoamphopropionate, stearoamphopropionate, lauroamphopropionate, salts thereof, and a mixture thereof.

### Nonionic Surfactants

In various embodiments, the compositions of the instant disclosure include one or more nonionic surfactants. Nonlimiting examples of useful nonionic surfactants include alkoxylated fatty alcohols, alkoxylated polyol esters, alkoxylated glycerides, glucosides, alkanolamides, sorbitan derivatives, or combinations thereof.

The total amount of the one or more nonionic surfactants in the cleansing composition may be from about 0.01 to about 10 wt. %, based on the total weight of the cleansing composition. In further embodiments, the cleansing composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more nonionic surfactants, based on the total weight of the cleansing composition.

Nonionic surfactants may optionally be alkoxylated. The alkoxylated nonionic surfactants may be chosen from alkoxylated alcohols, alkoxylated fatty alcohols, alkoxylated polyol esters such as polyethylene glycol ethers of fatty alcohols, polyethylene glycol ethers of esters, and polyethylene glycol ethers of glycerides, and mixtures thereof. Non-limiting examples of polyethylene glycol ethers of esters include ethoxylated fatty esters. Further discussion of non-limiting examples of the alkoxylated nonionic surfactants are provided below. In some instances, the alkoxylated nonionic surfactants are chosen from PEG-55 propylene glycol oleate, PEG-6 propylene glycol caprylate/caprate, PEG-8 propylene glycol cocoate, PEG-55 propylene glycol oleate, PEG-75 propylene glycol stearate, PEG-25 propylene glycol stearate, PEG-7 glyceryl cocoate, PEG-30 glyceryl cocoate, laureth-2, laureth-3, laureth-4, PEG-200 glyceryl stearate, PEG-120 propylene glycol stearate, PEG-6 Caprylic/Capric Glycerides, and a mixture thereof.

“Alkoxylated nonionic surfactant” as used herein means a compound having at least one alkoxylated portion (—(CH2)nO—, where n is an integer from 1 to 300, preferably 2 to 200, or more preferably 2 to 150, even more preferably 2 to 120, or most preferably, 2 to 100).

### (a) Alkoxylated Fatty Alcohols

“Alkoxylated fatty alcohol” as used herein means a compound having at least one fatty portion (8 carbon atoms or more) and at least one alkoxylated portion (—(CH2)nO—, where n is an integer of 1 or more). The alkoxylated fatty alcohols of the present invention preferably have an HLB (hydrophilic-lipophilic balance) value from 1-20, including all ranges and subranges therebetween, with HLB values ranging from 1 to 5 (particularly 3 to 5) or from 15-20 (particularly 16 to 18) being preferred. The alkoxylated fatty alcohol may be chosen from ethoxylated fatty alcohols, propoxylated fatty alcohols, and mixtures thereof.

The alkoxylated fatty alcohol can be chosen from di-alkyl, tri-alkyl- and combinations of di-alkyl and tri-alkyl substituted ethoxylated polymers. They can also be chosen from mono-alkyl, di-alkyl, tri-alkyl, tetra-alkyl substituted alkyl ethoxylated polymers and all combinations thereof. The alkyl group can be saturated or unsaturated, branched or linear and contain a number of carbon atoms preferably from about 12 carbon atoms to about 50 carbon atoms, including all ranges and subranges therebetween, for example, 20 to 40 carbon atoms, 22 to 24 carbon atoms, 30 to 50 carbon atoms, and 40 to 60 carbon atoms. Preferably, the fatty portion contains a mixture of compounds of varying carbon atoms such as, for example, C₂₀-C₄₀ compounds, C₂₂-C₂₄ compounds, C₃₀-C₅₀ compounds, and C₄₀-C₆₀ compounds.

Preferably, the alkoxylated portion of the alkoxylated fatty alcohols of the present disclosure contain 2 or more alkoxylation units, preferably from 2 to 20 alkoxylation units, preferably from 2 to 12 alkoxylation units, preferably from 10 to 200 alkoxylation units, preferably from 20 to 150 alkoxylation units, and preferably from 25 to 100 alkoxylation units, including all ranges and subranges therebetween. Also preferably, the alkoxylation units contain 2 carbon atoms (ethoxylation units) and/or 3 carbon atoms (propoxylation units).

The amount of alkoxylation can also be determined by the percent by weight of the alkoxylated portion with respect to the total weight of the compound. Suitable weight percentages of the alkoxylated portion with respect to the total weight of the compound include, but are not limited to, 10 percent to 95 percent, preferably 20 percent to 90 percent, including all ranges and subranges therebetween with 75 percent to 90 percent (particularly 80 percent to 90 percent) or 20 percent to 50 percent being preferred.

Preferably, the alkoxylated fatty alcohols of the present invention have a number average molecular weight (Mn) greater than 500, preferably from 500 to 5,000, including all ranges and subranges therebetween such as, for example, Mn of 500 to 1250 or an Mn of 2,000 to 5,000.

Suitable examples of alkoxylated fatty alcohols include: laureth-3, laureth-4, laureth-7, laureth-9, laureth-12, laureth-23, ceteth-10, steareth-10, steareth-2, steareth-100, beheneth-5, beheneth-5, beheneth-10, oleth-10, Pareth alcohols, trideceth-10, trideceth-12, C12-13 pareth-3, C12-13 pareth-23, C11-15 pareth-7, PEG hydrogenated castore oil, PEG-75 lanolin, polysorbate-80, polysobate-20, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and mixtures thereof.

### (b) Alkoxylated Polyol Esters

The alkoxylated polyol esters may be chosen from pegylated derivatives of propylene glycol oleate, propylene glycol caprylate/caprate, propylene glycol cocoate, propylene glycol stearate, and a mixture thereof. In certain embodiments, the alkoxylated polyol esters are chosen from PEG-55 propylene glycol oleate, PEG-6 propylene glycol caprylate/caprate, PEG-8 propylene glycol cocoate, PEG-25 propylene glycol stearate, and PEG-120 propylene glycol stearate, and a mixture thereof. In some instances, the polyol ester is or includes PEG-55 propylene glycol oleate. While the alkoxylated polyol esters comprise PEG-200 glyceryl stearate in some embodiments, in other embodiments PEG-200 glyceryl stearate may be excluded. Additionally and/or alternatively, the polyol esters may be chosen from ethoxylated fatty acid esters of sorbitan comprising from 2 to 30 mol of ethylene oxide.

In some cases, the polyol ester may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Sources of unsaturated polyol esters of glycerol include synthesized oils, natural oils (e.g., vegetable oils, algae oils, bacterial derived oils, and animal fats), combinations of these, and the like. Non-limiting examples of vegetable oils include Abyssinian oil, Almond oil, Apricot oil, Apricot Kernel oil, Argan oil, Avocado oil, Babassu oil, Baobab oil, Black Cumin oil, Black Currant oil, Borage oil, Camelina oil, Carinata oil, Canola oil, Castor oil, Cherry Kernel oil, Coconut oil, Corn oil, Cottonseed oil, Echium oil, Evening Primrose oil, Flax Seed oil, Grape Seed oil, Grapefruit Seed oil, Hazelnut oil, Hemp Seed oil, Jatropha oil, Jojoba oil, Kukui Nut oil, Linseed oil, Macadamia Nut oil, Meadowfoam Seed oil, Moringa oil, Neem oil, Olive oil, Palm oil, Palm Kernel oil, Peach Kernel oil, Peanut oil, Pecan oil, Pennycress oil, Perilla Seed oil, Pistachio oil, Pomegranate Seed oil, Pongamia oil, Pumpkin Seed oil, Raspberry oil, Red Palm Olein, Rice Bran oil, Rosehip oil, Safflower oil, Seabuckthorn Fruit oil, Sesame Seed oil, Shea Olein, Sunflower oil, Soybean oil, Tonka Bean oil, Tung oil, Walnut oil, Wheat Germ oil, High Oleoyl Soybean oil, High Oleoyl Sunflower oil, High Oleoyl Safflower oil, High Erucic Acid Rapeseed oil, combinations of these, and the like. Non-limiting examples of animal fats include lard, tallow, chicken fat, yellow grease, fish oil, emu oil, combinations of these, and the like. Non-limiting example of a synthesized oil includes tall oil, which is a byproduct of wood pulp manufacture. In some embodiments, the natural oil is refined, bleached, and/or deodorized.

The polyol esters may optionally be a natural polyol esters chosen from vegetable oil, an animal fat, an algae oil and mixtures thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, in one aspect, sucrose, and mixtures thereof.

Additional non-limiting examples of nonionic surfactants that may optionally be used in the cleansing composition include and/or may be chosen from alkanolamides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated oils from plant origin; fatty acid esters of sucrose; fatty acid esters of polyethylene glycol; N—(C₆-C₂₄)alkylglucamine derivatives, amine oxides such as (C₁₀-C₁₄)alkylamine oxides or N—(C₁₀-C₁₄) acylaminopropylmorpholine oxides; and mixtures thereof.

### (c) Alkoxylated Glycerides

Non-limiting examples of alkoxylated glycerides that may be suitable in certain embodiments include PEG-6 almond glycerides, PEG-20 almond glycerides, PEG-35 almond glycerides, PEG-60 almond glycerides, PEG-192 apricot kernel glycerides, PEG-11 avocado glycerides, PEG-14 avocado glycerides, PEG-11 babassu glycerides, PEG-42 babassu glycerides, PEG-4 caprylic/capric glycerides, PEG-6 caprylic/capric glycerides, PEG-7 caprylic/capric glycerides, PEG-8 caprylic/capric glycerides, PEG-11 cocoa butter glycerides, PEG-75 cocoa butter glycerides, PEG-7 cocoglycerides, PEG-9 cocoglycerides, PEG-20 corn glycerides, PEG-60 corn glycerides, PEG-20 evening primrose glycerides, PEG-60 evening primrose glycerides, PEG-5 hydrogenated corn glycerides, PEG-8 hydrogenated fish glycerides, PEG-20 hydrogenated palm glycerides, PEG-6 hydrogenated palm/palm kernel glyceride, PEG-16 macadamia glycerides, PEG-70 mango glycerides, PEG-13 mink glycerides, PEG-25 moringa glycerides, PEG-42 mushroom glycerides, PEG-2 olive glycerides, PEG-6 olive glycerides, PEG-7 olive glycerides, PEG-10 olive glycerides, PEG-40 olive glycerides, PEG-18 palm glycerides, PEG-12 palm kernel glycerides, PEG-45 palm kernel glycerides, PEG-60 Passiflora edulis seed glycerides, PEG-60 Passiflora incarnata seed glycerides, PEG-45 safflower glycerides, PEG-60 shea butter glycerides, PEG-75 shea butter glycerides, PEG-75 shorea butter glycerides, PEG-35 soy glycerides, PEG-75 soy glycerides, PEG-2 sunflower glycerides, PEG-7 sunflower glycerides, PEG-10 sunflower glycerides, PEG-13 sunflower glycerides, PEG-5 tsubakiate glycerides, PEG-10 tsubakiate glycerides, PEG-20 tsubakiate glycerides, PEG-60 tsubakiate glycerides, and sodium PEG-8 palm glycerides carboxylate.

In some embodiments, the at least one alkoxylated nonionic surfactant includes alkoxylated polyol esters such as polyethylene glycol ethers of esters. For example, the polyethylene glycol ethers of esters may be chosen from PEG-55 propylene glycol oleate, PEG-6 propylene glycol caprylate/caprate, PEG-8 propylene glycol cocoate, PEG-25 propylene glycol stearate, PEG-7 glyceryl cocoate, PEG-30 glyceryl cocoate, laureth-2, laureth-3, laureth-4, PEG-200 glyceryl stearate PEG-55 propylene glycol oleate. In further embodiments, the alkoxylated nonionic surfactants comprise a polyethylene glycol ethers of esters and at least one alkoxylated nonionic surfactant other than a polyethylene glycol ether of an ester.

In an embodiment, the at least one alkoxylated nonionic surfactant comprises at least one polyethylene glycol ether of fatty alcohols. For example, the polyethylene glycol ether of fatty alcohol may be chosen from laureth-2, laureth-3, laureth-4, steareth-20, or a mixtures thereof. The polyethylene glycol ether of fatty alcohols may have from 8 to 30 carbon atoms and in particular from 10 to 22 carbon atoms, such as polyethylene glycol ethers of cetyl alcohol, of stearyl alcohol or of cetearyl alcohol (mixture of cetyl alcohol and stearyl alcohol). Mention may be made, for example, of ethers including from 1 to 200 and preferably from 2 to 100 oxyethylene groups, such as those with the CTFA name Ceteareth-20 or Ceteareth-30, and mixtures thereof.

In an embodiment, the at least one alkoxylated nonionic surfactant comprises at least one polyethylene glycol ether of glycerides. For example, the polyethylene glycol ether of glyceride may be chosen from PEG-6 Caprylic/Capric Glycerides). In another embodiment, the cleansing composition comprises at least two alkoxylated nonionic surfactant. Preferably, one of the at least two alkoxylated nonionic surfactants is PEG-55 propylene glycol oleate.

Further nonionic surfactants that may optionally be present in the cleansing composition include:

### (d) Glucosides

The term glucoside is interchangeable with the term “alkyl polyglucoside.” In some embodiments, the one or more glucosides include those chosen from lauryl glucoside, octyl glucoside, decyl glucoside, coco glucoside, caprylyl/capryl glucoside, sodium lauryl glucose carboxylate, and a mixture thereof. Additionally or alternatively, the glucosides may be a alkyl polyglucoside that is chosen from glycerol (C₆-C₂₄)alkylpolyglycosides including, e.g., polyethoxylated fatty acid mono or diesters of glycerol (C₆-C₂₄)alkylpolyglycosides. Additional alkyl polyglucosides that may be suitably incorporated, in some instances, in the cleansing composition includes alkyl polyglucosides having a structure according to the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Alkyl poly glucosides may, in some instances, include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

### (e) Alkanolamides

Nonlimiting examples of alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one-OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides may include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

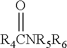

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof); wherein R₅ is selected from —CH₂OH, —CH₂CH₂OH, —CH₂CH₂CH₂OH, —CH₂ (CHOH)₄CH₂OH,-benzyl, and mixtures thereof; and wherein R₆ is selected from —H, —CH₃, —CH₂OH, —CH₂CH₃, —CH₂CH₂OH, —CH₂CH₂CH₂OH, —CH₂ (CHOH)₄CH₂OH,-benzyl, and mixtures thereof.

In some instances, the one or more of the fatty acid alkanolamides include one or more acyl glucamides, e.g., acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

### (f) Sorbitan Derivatives

Suitable sorbitan derivatives that may be incorporated into the plurality of nonionic surfactants include those chosen from polysorbate-20 (POE (20) sorbitan monolaurate), polysorbate-21 (POE (4) sorbitan monolaurate), polysorbate-40 (POE (20) sorbitan monopalmitate), polysorbate-60 (POE (20) sorbitan monostearate), polysorbate-61 (POE (4) sorbitan monostearate), polysorbate-65 (POE (20) sorbitan tristearate), polysorbate-80 (POE (20) sorbitan monooleate), polysorbate-81 (POE (4) sorbitan monooleate), polysorbate 85 (POE (20) Sorbitan Trioleate), sorbitan isostearate, sorbitan monolaurate, sorbitan monooleate, sorbitan monopalmitate, sorbitan monostearate, sorbitan sesquioleate, sorbitan trioleate and sorbitan tristearateand a mixture thereof.

Additional and/or alternative sorbitan derivatives include sorbitan esters including, e.g., esters of C₁₆-C₂₂ fatty acid and of sorbitan that were formed by esterification, with sorbitol, of at least one fatty acid comprising at least one saturated or unsaturated linear alkyl chain respectively having from 16 to 22 carbon atoms. These esters can be chosen in particular from sorbitan stearates, behenates, arachidates, palmitates or oleates, and their mixtures. Examples of optional sorbitan esters include sorbitan monostearate (INCI name: Sorbitan stearate) sold by Croda under the name Span 60, the sorbitan tristearate sold by Croda under the name Span 65 V, the sorbitan monopalmitate (INCI name: Sorbitan palmitate) sold by Croda under the name Span 40, the sorbitan monooleate sold by Croda under the name Span 80 V or the sorbitan trioleate sold by Uniqema under the name Span 85 V. A preferable sorbitan ester is sorbitan tristearate.

### Cationic Surfactants

The term “cationic surfactant” as used in the present disclosure is a surfactant that may be positively charged when it is contained in the hair treatment compositions according to the present disclosure. The cationic surfactant may bear one or more positive permanent charges or may contain one or more functional groups that are cationizable in the compositions.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

The total amount of the one or more cationic surfactants, present, will vary but may be in an amount from about 0.01 to about 10 wt. %, based on the total weight of the cleansing composition. In further embodiments, the cleansing composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the cleansing composition.

### (d) Water

The total amount of water in the cleansing composition will vary but is typically from about 40 to about 95 wt. % based on the total weight of the cleansing composition. In further embodiments, the total amount of water in the cleansing composition is from about 50 to about 95 wt. %, about 60 to about 95 wt. %, about 70 to about 95 wt. %, about 75 to about 95 wt. %, about 75 to about 93 wt. %, about 80 to about 95 wt. %, about 80 to about 93 wt. %, about 85 to about 95 wt. %, or about 85 to about 93 wt. %, based on the total weight of the final emulsion.

As mentioned above, in various embodiments, it is useful for the cleansing composition to include a large oil phase, which in turn reduces the amount of water needed for the cleansing composition. In this case, the total amount of water in the cleansing composition may be from about 30 to about 80 wt. %, based on the total weight of the cleansing composition. In further embodiments, the total amount of water in the cleansing composition may be from about 30 to about 70 wt. %, about 30 to about 60 wt. %, about 30 to about 50 wt. %, about 40 to about 70 wt. %, about 40 to about 60 wt. %, or about 40 to about 50 wt. %, based on the total weight of the cleansing composition.

### Water Soluble Solvent

The cleansing composition optionally includes one or more water soluble solvents. The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, mono-alcohols (for example C₂₋₈, or C₂₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof. In a preferred embodiment, the composition include one or more glycols selected from propylene glycol, butylene glycol, pentylene glycol, hexylene glycol, caprylyl glycol, dipropylene glycol, and mixtures thereof.

The total amount of the one or more water soluble solvents in the cleansing composition, if present, will vary. Nonetheless, the cleansing composition may include from about 0.1 to about 30 wt. % of one or more water soluble solvents, based on the total weight of the cleansing composition. In further embodiments, the cleansing composition includes from about 0.1 to about 20 wt. %, about 0.1 to about 15 wt. %, about 0.1 to about 10 wt. %, about 0.1 to about 5 wt. %, about 1 to about 30 wt. %, about 1 to about 30 wt. %, about 1 to about 15 wt. %, about 1 to about 10 wt. %, about 1 to about 5 wt. %, about 2 to about 30 wt. %, about 2 to about 20 wt. %, about 2 to about 15 wt. %, about 2 to about 10 wt. %, or about 2 to about 8 wt. % of one or more water soluble solvents, based on the total weight of the cleansing composition.

### Cationic Conditioning Polymers

The cleansing compositions of the instant disclosure may optionally include one or more cationic polymers. Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure, cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), or cationic guar (guar derivatized to be cationic).

Nonlimiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Nonlimiting examples of cationic guar include guar hydroxypropyltrimonium chloride, hydroxypropyl guar hydroxypropyltrimonium chloride, guar hydroxypropyltrimethylammonium chloride, and mixtures thereof.

Nonlimiting examples of cationic starch include starch hydroxypropyltrimonium chloride, hydroxypropyl oxidized starch PG trimonium chloride, and a mixture thereof.

In certain embodiments, the composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene (dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine) methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The total amount of the one or more cationic polymers in the cleansing composition, if present, will vary. Nonetheless, in certain embodiments, the cleansing composition includes about 0.01 to about 5 wt. % of one or more cationic conditioning polymers, based on the total weight of the cleansing composition. The compositions may include about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, based on the total weight of the cleansing composition.

### Thickening Agents

The cleansing composition may optionally include one or more thickening agents. Thickening agents that may be mentioned include the following:
a. Carboxylic acid or carboxylate based homopolymer or co-polymer, which can be linear or crosslinked: These polymers contain one or more monomers derived from acrylic acid, substituted acrylic acids, and salts and esters of these acrylic acids (acrylates) and the substituted acrylic acids. Commercially available polymers include those sold under the trade names CARBOPOL, ACRYSOL, POLYGEL, SOKALAN, CARBOPOL ULTREZ, and POLYGEL. Examples of commercially available carboxylic acid polymers include the carbomers, which are homopolymers of acrylic acid crosslinked with allyl ethers of sucrose or pentaerytritol. The carbomers are available as the CARBOPOL 900 series from B.F. Goodrich (e.g., CARBOPOL 954). In addition, other suitable carboxylic acid polymeric agents include ULTREZ 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL 1342, CARBOPOL 1382, PEMULEN TR-1, and PEMULEN TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is available in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw maerial known under the tradename of SIMULQUAT HC 305 from Seppic.

In certain embodiments, the carboxylic acid or carboxylate polymer thickeners useful herein are those selected from carbomers, acrylates/C10-C30 alkyl acrylate crosspolymers, polyacrylate crosspolymer-6, acrylamidopropyltrimonium chloride/acrylates copolymer, and mixtures thereof.
b. Celluloses: Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some instances, the cellulose is selected from water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is preferably hydroxypropylcellulose (HPC).
c. Polyvinylpyrrolidone (PVP) and co-polymers: Non-limiting examples include Polyvinylpyrrolidone (PVP), Polyvinylpyrrolidone (PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes LUVISKOL K30, K85, K90 available from BASF. Commerically available copolymers of vinylpyrrolidone and vinylacetate include LUVISKOL VA37, VA64 available from BASF; copolymers of vinylpyrrolidone, methacrylamide, and vinylimidazole (INCI: VP/Methacrylamide/Vinyl Imidazole Copolymer) is commercially available as LUVISET from BASF. In some instances, PVP and PVP/VA copolymer are preferred.
d. Sucrose esters: Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.
e. Polyglyceryl esters: Non-limiting polyglycerol esters of fatty acids (polygylceryl esters) include those of the following formula:

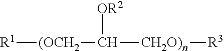

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.
f. C8-24 hydroxyl substituted aliphatic acid and C8-24 conjugated aliphatic acid: Non-limiting examples include conjugated linoleic acid, cis-parinaric acid, trans-7-octadecenoic acid, cis-5,8,11,14,17-eicosapentanoic acid, cis-4,7,10,13,16,19-docosahexenoic acid, columbinic acid, linolenelaidic acid, ricinolaidic acid, stearidonic acid, 2-hydroxystearic acid, alpha-linolenic acid, arachidonic acid, cis-11,14-eicosadienoic acid, linolelaidic acid, monopetroselinic acid, petroselinic acid, ricinoleic acid, trans-vaccenic acid, cis-11,14,17-eicosatrienoic acid, cis-5-eicosenoic acid, cis-8,11,14-eicosatrienoic acid, hexadecatrienoic acid, palmitoleic acid, petroselaidic acid, trans trans farnesol, cis-13,16-docosadienoic acid, cis-vaccenic acid, cis-11-eicosenoic acid, cis-13,16,19-docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, pinolenic acid, trans-13-octadecenoic acid. More preferably, the aliphatic acid comprises 12-hydroxystearic acid, conjugated linoleic acid, or a mixture thereof.
g. Gums: Non-limiting examples of gums include gum arabic, tragacanth gum, karaya gum, guar gum, gellan gum, tara gum, locust bean gum, tamarind gum, xanthan gum, locust bean gum, Seneca gum, sclerotium gum, gellan gum, etc.

Nonlimiting examples of thickening agent that may optionally be used include polymers, gums, organoclays, polyethylenes, silica, for example, acrylate copolymer, hectorite gel, silica, silica dimethyl silylate, behenate, glyceryl dibehenate, behenyl behenate, or mixtures thereof.

The total amount of one or more thickening agent, if present, will but may be in an amount from about 0.01 to about 6 wt. %, based on the total weight of the cosmetic composition. In further embodiments, the total amount of the one or more thickening agents may be from about 0.01 to about 4 wt. %, about 0.01 to about 3 wt. %, about 0.1 to about 6 wt. %, about 0.1 to about 5 wt. %, about 0.1 to about 4 wt. %, about 0.1 to about 3 wt. %, about 0.5 to about 6 wt. %, about 0.5 to about 5 wt. %, about 0.5 to about 4 wt. %, about 0.5 to about 3 wt. %, about 1 to about 5 wt. %, or about 1 to about 3 wt. %, based on the total weight of the cleansing composition.

pH

The pH of the cleansing composition is typically less than 7. For example, the pH of the cleansing composition may be from about 4 to less than 7, about 4.5 to less than 7, about 5 to less than 7, about 5.5 to less than 7, about 6 to less than 7, about 4 to about 6.5, about 4.5 to about 6.5, about 5 to about 6.5, or about 5.5 to about 6.5.

### Aqueous Phase

The cleansing compositions are in the form of an oil-in-water emulsion having an aqueous phase and an oil phase. The aqueous phase may constitute about 10 to about 90 wt. % of the cleansing composition, based on the total weight of the cleansing composition. In further embodiments, the aqueous phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. % of the cleansing composition, based on the total weight of the cleansing composition.

### Oil Phase

The oil phase may constitute about 10 to about 90 wt. % of the cleansing composition, based on the total weight of the cleansing composition. In further embodiments, the oil phase may constitute about 10 to about 80 wt. %, about 10 to about 70 wt. %, about 10 to about 60 wt. %, about 10 to about 50 wt. %, about 10 to about 40 wt. %, about 10 to about 30 wt. %, about 20 to about 90 wt. %, about 20 to about 80 wt. %, about 20 to about 70 wt. %, about 20 to about 60 wt. %, about 20 to about 50 wt. %, about 20 to about 40 wt. %, about 20 to about 30 wt. %, about 30 to about 70 wt. %, about 30 to about 60 wt. %, or about 30 to about 40 wt. %, based on the total weight of the cleansing composition.

### Droplet Size

In various embodiments, the average droplet size of the oil phase in the emulsions forming the cleansing composition is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

Droplet size can be determined using Brookhaven Dynamic Light Scattering (DLS). DLS is a technique used to determine droplet size in a colloidal system or emulsion. When the samples are illuminated with a monochromatic laser beam, the particles in the samples undergo Brownian motion, causing fluctuations in scattered light intensity. The scattered light is then collected at various angles, and the autocorrelation function of these intensity fluctuations is analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### EMBODIMENTS

In a preferred embodiment, the cleansing wipe comprises, consists essentially of, or consists of:
(i) a insoluble flexible substrate; and
(ii) a cleansing composition, wherein the cleansing composition comprises:
   (a) about 0.1 to about 8 wt. %, preferably about 0.1 to about 5 wt. %, more preferably about 0.5 to about 3 wt. % of a hydrophobic polymer, which is a reaction product of a natural or food-derived oil and a methacrylate or acrylate polymer, wherein preferably,
      the hydrophobic polymer is a reaction product of: (a) (i) a natural or food-derived oil selected from linseed oil, sunflower oil, tung oil, fish oil, cottonseed oil, soybean oil, or combinations thereof, preferably linseed oil, and (a) (ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
      the hydrophobic polymer is the reaction product of linseed oil and poly(isobutyl methacrylate);
(a) about 0.1 to about 10 wt. %, preferably about 0.1 to about 6 wt. %, more preferably about 0.5 to about 5 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 15 wt. %, preferably about 0.5 to about 10 wt. %, more preferably about 1 to about 8 wt. % of one or more surfactants, preferably a plurality of surfactants;
(d) about 75 to about 95 wt. %, preferably about 80 to about 95 wt. %, more preferably about 85 to about 93 wt % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the cleansing composition is an oil-in-water emulsion, and
   the substrate is impregnated with the cleaning composition.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cleansing composition is typically less than 7. For example, the pH of the cleansing composition may be from about 4 to less than 7, about 4.5 to less than 7, about 5 to less than 7, about 5.5 to less than 7, about 6 to less than 7, about 4 to about 6.5, about 4.5 to about 6.5, about 5 to about 6.5, or about 5.5 to about 6.5.

In another preferred embodiments, the cleansing wipe comprises, consists essentially of, or consists of:
(i) a insoluble flexible substrate; and
(ii) a cleansing composition, wherein the cleansing composition comprises:
   (b) about 0.1 to about 8 wt. %, preferably about 0.1 to about 5 wt. %, more preferably about 0.5 to about 3 wt. % of a hydrophobic polymer, which is a reaction product of a natural or food-derived oil and a methacrylate or acrylate polymer, wherein preferably,
      the hydrophobic polymer is a reaction product of: (a) (i) a natural or food-derived oil selected from linseed oil, sunflower oil, tung oil, fish oil, cottonseed oil, soybean oil, or combinations thereof, preferably linseed oil, and (a) (ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
      the hydrophobic polymer is the reaction product of linseed oil and poly(isobutyl methacrylate);
   (b) about 5 to about 50 wt. %, preferably about 10 to about 45 wt. %, more preferably about 15 to about 40 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 15 wt. %, preferably about 0.5 to about 10 wt. %, more preferably about 1 to about 8 wt. % of one or more surfactants, preferably a plurality of surfactants, wherein preferably the plurality of surfactants include:
(c) (i) about 0.1 to about 10 wt. %, preferably about 0.5 to about 8 wt. %, more preferably about 1 to about 5 wt. % of one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and
(c) (ii) about 0.1 to about 10 wt. %, preferably about 0.5 to about 8 wt. %, more preferably about 1 to about 10 wt. % of one or more co-surfactants, preferably wherein the one or more additional co-surfactants are one or more anionic surfactants, one or more nonionic surfactants, one or more amphoteric surfactants or combination thereof, wherein even more preferably, the one or more co-surfactants include at least one anionic surfactants selected from sulfate surfactants (lauryl sulfate, sodium laureth ether sulfate, etc.), sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof, even more preferably an acyl taurate selected from sodium cocoyl taurate, sodium methyl cocoyl taurate, sodium lauroyl taurate, and sodium methyl lauroyl taurate, or mixtures thereof;
(d) about 45 to about 90 wt. %, preferably about 55 to about 85 wt. %, more preferably about 60 to about 80 wt. % of water;
(e) optionally, one or more water soluble solvents, preferably wherein the one or more water soluble solvents, if present, is selected from mono-alcohols (for example C₂-8, or C₂-4 alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the cleansing composition is an oil-in-water emulsion, and
   the substrate is impregnated with the cleaning composition.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cleansing composition is typically less than 7. For example, the pH of the cleansing composition may be from about 4 to less than 7, about 4.5 to less than 7, about 5 to less than 7, about 5.5 to less than 7, about 6 to less than 7, about 4 to about 6.5, about 4.5 to about 6.5, about 5 to about 6.5, or about 5.5 to about 6.5.

In another preferred embodiments, the cleansing wipe comprises, consists essentially of, or consists of:
(i) a insoluble flexible substrate; and
(ii) a cleansing composition, wherein the cleansing composition comprises:
   (a) about 0.1 to about 8 wt. %, preferably about 0.1 to about 5 wt. %, more preferably about 0.5 to about 3 wt. % of a hydrophobic polymer that is a reaction product of linseed oil and poly(isobutyl methacrylate);
   (b) one or more solvents capable of solubilizing the reaction product of (a), wherein the one or more solvents form a liquid oil phase, wherein preferably,
      the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 15 wt. %, preferably about 0.5 to about 10 wt. %, more preferably about 1 to about 8 wt. % of one or more surfactants, preferably a plurality of surfactants, wherein preferably the plurality of surfactants include:
(c) (i) one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and
(c) (ii) one or more co-surfactants, preferably wherein the one or more additional co-surfactants are one or more anionic surfactants selected from sulfate surfactants (lauryl sulfate, sodium laureth ether sulfate, etc.), sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof, even more preferably an acyl taurate selected from sodium cocoyl taurate, sodium methyl cocoyl taurate, sodium lauroyl taurate, and sodium methyl lauroyl taurate, or mixtures thereof;
(d) water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %;
(f) optionally, about 0.01 to about 8 wt. %, preferably about 0.1 to about 6 wt. %, more preferably about 0.5 to about 5 wt. % of one or more one or more thickening agents;
(g) optionally, from about 0.01 to about 6 wt. %, preferably from about 0.05 to about 5 wt. %, more preferably about 0.1 to about 3 wt. % of one or more skin active agents; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the cleansing composition has an oil phase and a water phase and is an oil-in-water emulsion, preferably an oil-in-water dispersion;
   the oil phase and the water phase are in a weight ratio of about 1:10 to about 10:1, preferably from about 1:5 to about 5:1, more preferably from about 1:3 to about 3:1, and wherein even more preferably, the oil phase and the water phase are in a weight ratio of about 1:1 to about 1:5 (oil phase:water phase);
   the substrate is impregnated with the cleaning composition.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cleansing composition is typically less than 7. For example, the pH of the cleansing composition may be from about 4 to less than 7, about 4.5 to less than 7, about 5 to less than 7, about 5.5 to less than 7, about 6 to less than 7, about 4 to about 6.5, about 4.5 to about 6.5, about 5 to about 6.5, or about 5.5 to about 6.5.

### EXAMPLES

Implementation of the present disclosure is provided by way of the following examples. The following examples serve to elucidate aspects of the technology without being limiting in nature.

### Example 1

The compositions illustrated in the table below were prepared. The compositions were prepared by first mixing the MycelX®, m-rhamnolipid, sodium methyl cocoyl taurate, and solvent (caprylic/capric triglyceride) with a small amount of water and mixing at 2,500 rpm for 2 minutes at 25° C. to form a homogenous mixture. The homogenous mixture was then diluted with additional water to form an oil-in-water emulsion.

| | Inventive | Comparative |
|---|---|---|
| INGREDIENTS | A | B |
| MycelX ®¹ | 2.6 | |
| m-Rhamnolipid | 3.2 | 3.2 |
| Sodium cocoyl Taurate | 1.3 | 1.3 |
| CAPRYLIC/CAPRIC | 0.6 | 0.6 |
| TRIGLYCERIDE | | |
| water QS | 100 | 100 |
| Percent Makeup Removal | 33% | 32% |

¹Reaction product of linseed oil and isobutyl methacrylate polymer

A commercially available liquid makeup foundation for application to the face was applied to an artificial skin template, i.e., to black supplare substrates (Miyoshi, Japan). An amount of 20 microliters of the liquid makeup foundation was uniformly applied to the substrates (20 microliter/9 cm²). The liquid makeup foundation was allowed to dry for 24 hours at 23-25° C. After 24 hours, L*a*b* values were determined for the supplare substrates coated with the makeup using a Datacolor 600™ Spectraflash spectrometer. The L-values were recorded. The L-value is a measure of the lightness of the sample. The higher the L-value, the whiter/brighter the sample.

An amount of about 1.5 ml of the inventive cleansing composition A and the comparative composition B were applied to different cotton pads having a radius of about 2-3 cm. The black supplare substrates coated with the makeup were then wiped with the cotton pads. The wiping was carried out under controlled pressure using a pressure sensor control at a pressure of about 300 gram force. After wiping, the black supplare substrates were allowed to dry at 23-25° C. Once dry, L*a*b* values were again determined, and the L-values noted. As makeup is removed, the black supplare substrate is revealed resulting in a lower L-Value. The makeup foundation is lighter (higher L value) than the black supplare substrates and therefore the more makeup present on the substrate, the higher the L-value. The percentage removal of the makeup foundation was determined as follows:

$$\% removal = ( {L}_{before} - {L}_{after} ) / ( {L}_{before} - {L}_{supplare} )$$

The results are reported in the table above and show that the inventive composition A, which included MycelX®, provided greater removal of the liquid foundation from the black supplare substrates.

### Example 2

Inventive compositions C and D in the table below were prepared like the compositions in Example 1. The “benchmark” is a commercially available cleansing product advertised for use in removing makeup from the skin.

| | Inventive | | Benchmark |
|---|---|---|---|
| INGREDIENTS | C | D | E |
| MycelX ®¹ | 2.6 | 2.6 | 2.6 |
| m-Rhamnolipid | 3.2 | 3.2 | 3.2 |
| Sodium cocoyl Taurate | 1.3 | 1.3 | 1.3 |
| Polycitronell Acetate | 0.6 | | |
| Isododecane | | 0.6 | |
| water QS | 100 | 100 | 100 |
| Percent Makeup Removal | 24% | 25% | 10% |

¹Reaction product of linseed oil and isobutyl methacrylate polymer

A commercially available liquid makeup foundation for application to the face was applied to an artificial skin template, i.e., a black supplare substrates (Miyoshi, Japan). An amount of 20 microliters of the liquid makeup foundation (20 microliter/9 cm²) was uniformly applied to the surface of the substrates. The liquid makeup was allowed to dry on the black supplare substrates for 24 hours at 23-25° C. After 24 hours, L*a*b* values were determined for the supplare substrates coated with the dry makeup, using a Datacolor 600™ Spectraflash spectrometer. The L-values were recorded. The L-value is a measure of the lightness of the sample. The higher the L-value, the whiter/brighter the sample.

An amount of about 1.5 ml of the inventive composition C or D, or the benchmark product, were applied to different cotton pads having a radius of about 2-3 cm. The black supplare substrates coated with makeup were then wiped with the cotton pads. The wiping was carried out under controlled pressure using a pressure sensor control with a pressure of about 300 gram force. After wiping, the black supplare substrates were allowed to dry at 23-25° C. Once dry, L*a*b* values were again assessed, and the L-values noted (more Makeup-higher L value). As makeup is removed, the black (low L value) supplare substrate is revealed resulting in a lower L-Value. The percentage removal of the makeup from the substrates was determined as follows:

$$\% removal = ( {L}_{before} - {L}_{after} ) / ( {L}_{before} - {L}_{supplare} )$$

The results are reported in the table above and show that inventive compositions C and D, which included MycelX®, provided much greater removal of makeup than the commercial benchmark.

### Example 3

### Foam Boosting

Testing was carried out to determine how MycelX® influences foaming. The composition set forth below were prepared according to the inventive procedure described in Example 2, i.e., a concentrated first composition was prepared followed by dilution.

| | Inventive | | | Comp. |
|---|---|---|---|---|
| INGREDIENTS | 1 | 2 | 3 | 4 |
| MYCELX ®¹ | 0.6 | 0.6 | 5.2 | 0 |
| Rhamnolipid | 0.7 | 0.7 | 5.7 | 0.7 |
| SODIUM METHYL | 0.3 | 0.3 | 2.3 | 0.3 |
| COCOYL TAURATE | | | | |
| POLYCITRONELLOL | | 0.1 | | |
| ACETATE | | | | |
| CAPRYLIC/CAPRIC | 0.1 | | | 0.1 |
| TRIGLYCERIDE | | | | |
| Initial Foam Volume (mL) | 59.4 | 47.0 | 56.2 | 31.1 |
| Final Foam Volume (mL) | 47.8 | 38.0 | 40.1 | 26.6 |

¹Reaction product of linseed oil and isobutyl methacrylate polymer

A Kruss Dynamic Foam Analyzer (DFA100) was used to measure the foamability of the compositions at a temperature of 25° C. The device uses a accurately controlled foaming process and an optical sensor that measure the quantity (volume) of foam produced and the decay characteristic of the foam, i.e., the lastingness of the foam. The results reported in the table above show that both initial foam volume and final foam volume was significantly greater for the inventive compositions containing MycelX®.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

The term “translucent” with respect to a translucent composition indicates that the composition has a transmittance of at least 50% at a wavelength of 600 nm, for example measured using a Lambda 40 UV-visible spectrometer.

Other than in the operating examples, or where otherwise indicated, all amount expressing quantities of ingredients and/or reaction conditions may be modified in all instances by the term “about,” meaning within +/−5% of the indicated number. Thus, for a range of “about 1 to about 10 wt. %,” The lower amount of “about 1 wt. %” may extend down to 0.95 wt. %, which is 5% less than 1 wt. %. The higher amount of “about 10 wt. %” may extend up to 10.5 wt. %, which is 5% higher than 10 wt. %, i.e., a range of “0.95 wt. % to 10.5 wt. %.”

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both oily solvent and a surfactant. If a particular composition includes both an oily solvent and a surfactant, a single compound will serve as only the oily solvent or only as the surfactant (the single compound does not simultaneously serve as both the oily solvent and the surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. For example, if a composition is essentially free from compound X, the composition includes less that 2 wt. % of compound X, or less than 1 wt. % of compound X, or less than 0.5 wt. % of compound X, or less than 0.1 wt. % of compound X, or is free from compound X.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cleansing wipe comprising:
(i) an insoluble flexible substrate; and
(ii) a cleansing composition, wherein the cleansing composition comprises:
(a) a hydrophobic polymer formed as a reaction product of a natural or food-derived oil and a methacrylate or acrylate polymer;
(b) one or more solvents capable solubilizing the hydrophobic polymer of (a);
(c) one or more surfactants; and
(d) water;
wherein the cleansing composition is an oil-in-water emulsion, and
the substrate is impregnated with the cleaning composition.

2. The composition of claim 1, wherein the hydrophobic polymer is the reaction product of a natural or food-derived oil and a methacrylate polymer.

3. The composition of claim 1, wherein the natural or food-derived oil is a drying oil or semi-drying oil selected from linseed oil, sunflower oil, tung oil, fish oil, cottonseed oil, soybean oil, or combinations thereof.

4. The composition of claim 1, wherein the polymer is derived from monomers selected from isobutyl methacrylate, methyl methacrylate, ethyl methacrylate, n-butyl methacrylate, or combinations thereof.

5. The composition of claim 1, wherein the hydrophobic polymer is the reaction product of about 50 to about 85 parts by weight of the natural or food-derived oil and about 15 to about 50 parts by weight of the methacrylate or acrylate polymer.

6. The composition of claim 1, wherein the hydrophobic polymer is the reaction product of about 72 to about 77% of linseed oil and about 23 to about 28% of isobutyl methacrylate polymer.

7. The composition of claim 1, wherein the polymer is derived from one or more monomers selected from isobutyl methacrylate, methyl methacrylate, ethyl methacrylate, n-butyl methacrylate, or combinations thereof.

8. The composition of claim 1, wherein the polymer is isobutyl methacrylate polymer.

9. The composition of claim 1, wherein the hydrophobic polymer is the reaction product of linseed oil and poly(isobutyl methacrylate).

10. The composition of claim 1, wherein the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameter, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

11. The composition of claim 1, wherein at least one of the one or more solvents capable of solubilizing the reaction product of (a) is selected from polycitronellol acetate, caprylic/capric triglyceride, isododecane, isohexadecane, tetradecane, isopropyl myristate, isopropyl alcohol, octyldodecanol, ethanol, castor oil, and mixtures thereof.

12. The composition of claim 1, wherein at least one of the one or more surfactants is a biosurfactant, wherein the biosurfactant is a glycolipid selected from sophorolipids, rhamnolipids, trehalose lipids, mannosylerythritol lipids, or combinations thereof.

13. The composition of claim 1, wherein the one or more surfactants are selected from anionic surfactants, nonionic surfactants, amphoteric surfactants, or combinations thereof.

14. The composition of claim 13 comprising one or more anionic surfactants, wherein the one or more anionic surfactants are selected from sulfate surfactants, acyl glutamates, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof.

15. The composition of claim 14, wherein at least one of the one or more anionic surfactants is an acyl taurate.

16. The composition of claim 1 comprising a plurality of surfactants, wherein the plurality of surfactants include:
(c) (i) about 1 to about 8 wt. % of one or more rhamnolipids; and
(c) (ii) about 0.5 to about 5 wt. % of one or more anionic surfactants.

17. The composition of claim 1, wherein the oil-in-water emulsion is a dispersion having a droplet size from about 10 nm to about 1 μm.

18. A cleansing wipe comprising:
(i) an insoluble flexible substrate; and
(ii) a cleansing composition, wherein the cleansing composition comprises:
(a) about 0.1 to about 8 wt. % of a hydrophobic polymer that is a reaction product of linseed oil and poly(isobutyl methacrylate);
(b) about 0.1 to about 40 wt. % of a liquid oil solvent capable of solubilizing the hydrophobic polymer of (a);
(c) about 1 to about 10 wt % of a plurality of surfactants, wherein the plurality of surfactants comprises:
(c) (i) about 1 to about 8 wt. % of one or more rhamnolipids; and
(c) (ii) about 0.5 to about 5 wt. % of one or more so-surfactants;
(d) about 60 to about 95 wt. % of water;
wherein the cleaning composition is an oil-in-water dispersion with oil droplets having an average droplet size from about 10 nm to about 1 μm,
all weight percentages are based on the total weight of the cleansing composition, and
the substrate is impregnated with the cleaning composition.

19. A method of cleaning a surface comprising contacting the surface with the cleansing wipe of claim 1.

20. A method for cleansing makeup from the skin comprising contacting makeup present on the skin with the cleansing wipe of claim 1 and removing the makeup from the skin.
